(19) United States
(12) Patent Application Publication
(10) Pub. No.: US 2015/0251921 A1
(43) Pub. Date: Sep. 10, 2015

(54) FOOD WASTE COLLECTOR SYSTEM WITH TREATMENT OF RECIRCULATING WATER TO REDUCE BACTERIA LEVELS

(71) Applicant: Emerson Electric Co., St. Louis, MO (US)

(72) Inventors: William SOBANSKI, Kenosha, WI (US); Thomas R. BERGER, Racine, WI (US)

(73) Assignee: Emerson Electric Co., St. Louis, MO (US)

(21) Appl. No.: 14/637,497

(22) Filed: Mar. 4, 2015

Related U.S. Application Data
(60) Provisional application No. 61/950,234, filed on Mar. 10, 2014.

(51) Int. Cl. C02F 1/00; A61L 2/08; C02F 1/72; A61L 2/10; C02F 1/78; C02F 1/32

(52) U.S. Cl. CPC C02F 1/008; C02F 1/78; C02F 1/32; C02F 1/725; A61L 2/10; A61L 2/088; C02F 2103/002

(57) ABSTRACT

A food waste collector system includes a tank and a pump that recirculates water by pumping it from the tank and back into the tank, and a bacteria treatment method and system that treats the recirculating water to reduce bacteria. In an aspect, the bacteria treatment includes irradiation of recirculating water by ultraviolet light to sterilize bacteria. In an aspect, the bacteria treatment includes killing bacteria in the recirculating water with oxidizers. In an aspect, the bacteria treatment includes killing bacteria in the recirculating water with ozone. In an aspect, the food waste collector system includes an ultraviolet (UV) tube holder in which one or more UV tubes are disposed and through which the recirculating water is pumped. In an aspect, the UV tube holder has an inner wall surface with a catalytic surface coating that in conjunction with the UV light provides an oxidation process that generates the oxidizers.

### CROSS-REFERENCE TO RELATED APPLICATIONS

This application claims the benefit of U.S. Provisional Application No. 61/950,234 filed on Mar. 10, 2014. The entire disclosure of the above application is incorporated herein by reference.

### FIELD

The present disclosure relates to food waste collector systems, and more particularly, to a food waste collector system with treatment of recirculating water to reduce bacteria levels in the recirculating water.

### BACKGROUND

This section provides background information related to the present disclosure which is not necessarily prior art.

Food waste collector systems are typically used in commercial kitchens in lieu of food waste disposers. In a typical prior art food waste collector system, a recirculating stream of warm water (for example, 107° F.) is used to rinse food waste from plates prior to dishwashing. The food waste is collected in a perforated container (such as a strainer basket) that is received in the recirculation tank for the recirculating stream of water. Water in the recirculation tank is pumped by a pump to a spout that is disposed above the recirculation tank and discharges into it. The dishes are held under the spout and rinsed by the water being discharged from the spout. The perforated container can be removed from the recirculation tank, drained and the food waste collected in it emptied.

It has been found that the recirculating water in these types of food waste collector systems may possibly harbor higher than normal levels of bacteria such as E coli, salmonella and listeria compared to tap water from a water utility.

### SUMMARY

This section provides a general summary of the disclosure, and is not a comprehensive disclosure of its full scope or all of its features.

A food waste collector system in accordance with an aspect of the present disclosure includes a tank and a pump that recirculates water by pumping it from the tank and back into the tank, and a bacteria treatment method and system that treats the recirculating water to reduce bacteria levels therein. In an aspect, the bacteria treatment method and system includes irradiation of recirculating water by ultraviolet light to sterilize bacteria. In an aspect, the bacteria treatment system method and system includes killing bacteria in the recirculating water with oxidizers. In an aspect, the bacteria treatment method and system includes killing bacteria in the recirculating water with ozone.

In an aspect, the food waste collector system in accordance with an aspect of the present disclosure includes a tank, a pump and an ultraviolet (UV) tube holder in which one or more UV tubes are disposed. Water is recirculated from the tank by being pumped by the pump through the UV tube holder and then discharged back into the tank. The water flowing past the UV tubes in the UV tube holder is irradiated with UV light from the UV tubes which treats the water to reduce bacteria in it by sterilizing bacteria in the water making the bacteria unable to reproduce, which reduces bacterial levels in the water. In an aspect, a perforated container is removably receivable in the tank.

In an aspect, the UV tube holder includes an inner wall with a catalytic surface coating that in conjunction with the UV light provides an oxidation process that generates oxidizers that kill bacteria in the water to further treat the water to reduce bacteria in it. In an aspect, the catalytic surface coating is a titanium dioxide surface coating.

In an aspect, the food waste collector system includes an ozone generator from which ozone is injected into the recirculating water to treat the water to reduce bacteria by killing bacteria in the water. In an aspect, the ozone is injected into the water in the tank.

In an aspect, to further reduce the possibility of contamination, in addition to any or all of the foregoing treatment processes, the food waste collector system periodically replaces a portion of the water being recirculated with fresh water via a fresh water inlet. In an aspect, a controllable valve coupled to a source of fresh water is cycled open and closed by a controller coupled to the controllable valve to periodically inject fresh water into the water being recirculated.

In an aspect, a waterfall type spout through which the water is discharged back into the tank is coupled to an outlet of the ultraviolet tube holder.

In an aspect, the food waste collector system includes a soap injector that injects soap into the recirculating water.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a side perspective view of a food waste collector system in accordance with an aspect of the present disclosure;

FIG. 2 is a front perspective view of a portion of the food waste collector system of FIG. 1;

FIG. 3 is broken away side perspective view of a portion of the food waste collector system of FIG. 1;

FIG. 4 is a broken away bottom perspective view of the food waste collector system of FIG. 1; and

FIG. 5 is a side perspective view of a variation of the food waste collector system of FIG. 1.

Corresponding reference numerals indicate corresponding parts throughout the several views of the drawings.

### DETAILED DESCRIPTION

Example embodiments will now be described more fully with reference to the accompanying drawings.

With reference to the drawings, a food waste collector system 100 in accordance with an aspect of the present disclosure is shown. Food waste collector system 100 includes a tank 102 in which a perforated container 104 having an open top end 106 is removably received. Perforated container 104 could, for example, be a strainer basket. It should be understood that perforated container 104 could include a plurality of nested strainer baskets having different sizes of perforations. For example, perforated container 104 could have three nested strainer baskets with the inner basket having the largest size perforations and the outer basket having the smallest size perforations.

In the illustrative embodiment shown in the drawings, tank 102 is a cylindrical tank and perforated container 104 is illustratively a cylindrical container having a sidewall 108, a bottom (not shown) along with open top end 106. Illustratively, sidewall 108 includes perforations 110 therein as does the bottom of perforated container 104.

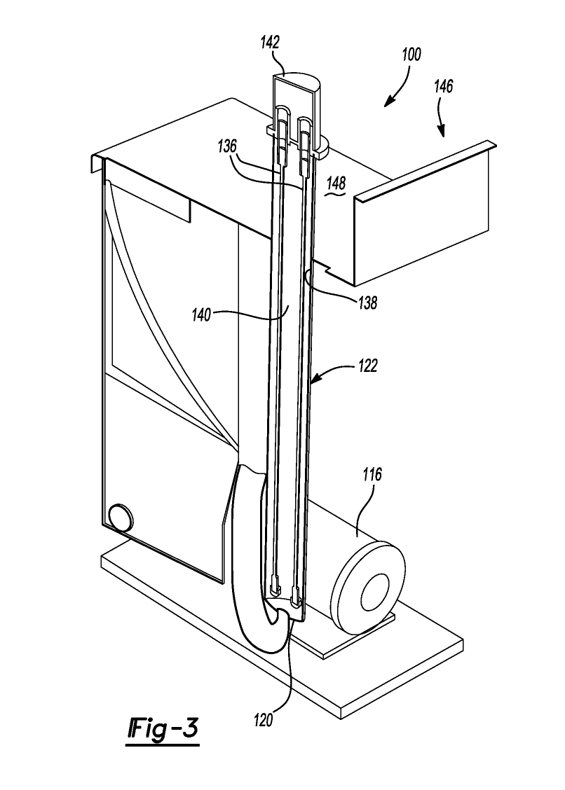

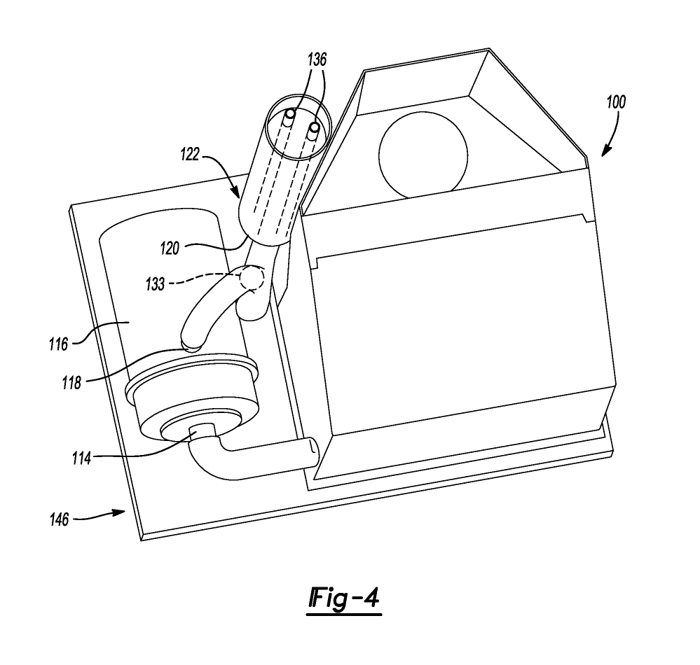

Tank 102 includes a water outlet 112 coupled to an inlet 114 of a pump 116. An outlet 118 of pump 116 is coupled through an optional filter (not shown) and a flow valve 133 to an inlet 120 of a UV tube holder 122 in which one or more ultraviolet (UV) tubes 136 (FIGS. 3 and 4) are disposed. In an aspect, an outlet 124 of UV tube holder 122 is coupled to a spout 126 that discharges into perforated container 104 when perforated container 104 is received in tank 102. In this regard, spout 126 provides an outlet through which the water being recirculated is discharged back into tank 102. Spout 126 may for example be a waterfall type spout providing a relatively flat, wide discharge stream of water. It should be understood that spout 126 provides an outlet Food waste collector system 100 also includes a fresh water inlet 128 coupled to a source of water (not shown), which may illustratively be a source of warm water. A controllable valve 132 (which may be a solenoid valve) is coupled in series between fresh water inlet 128 and tank 102.

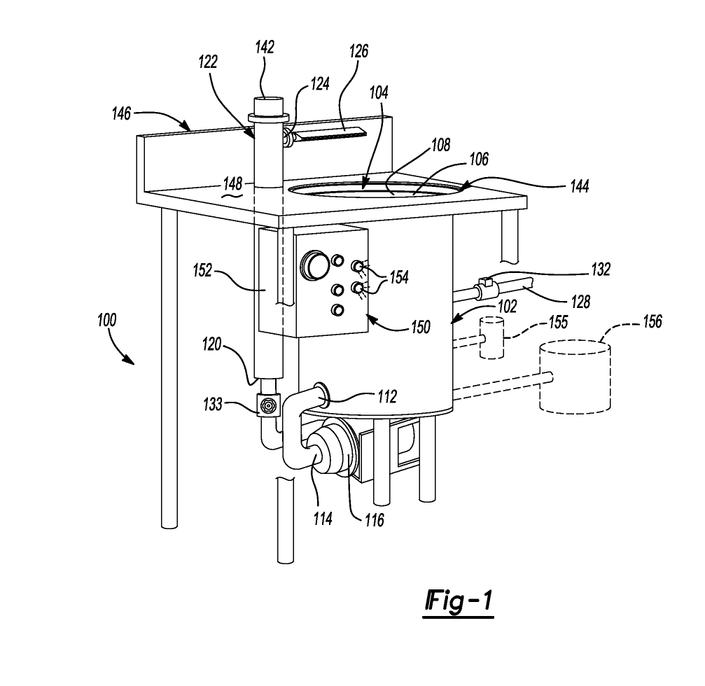

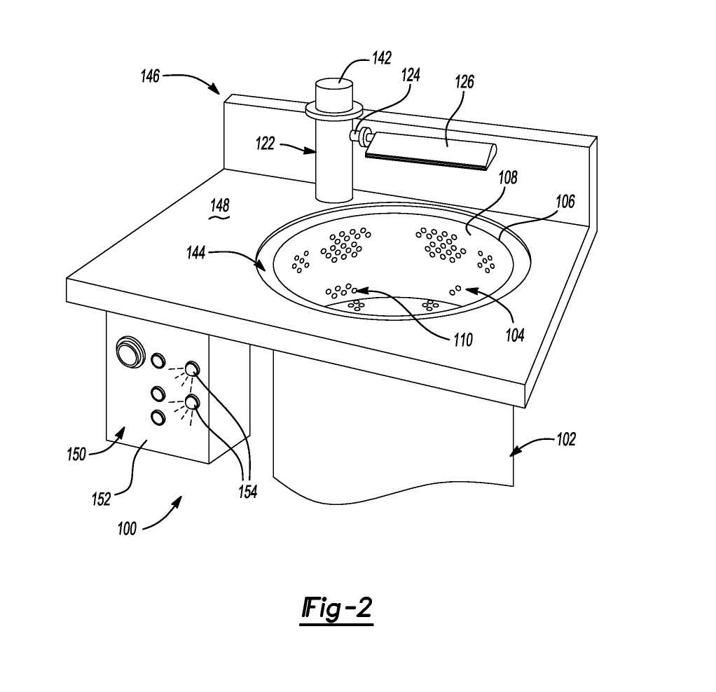

UV tube holder 122 may illustratively by a cylindrical tube. An inner wall 138 of UV tube holder 122 illustratively includes a catalytic surface coating 140 that reacts with ultraviolet light to provide an oxidation process that generates oxidizers. Catalytic surface coating 140 is illustratively a titanium dioxide surface coating, but could be other types of catalysts that react with ultraviolet light to enhance oxidation. UV tube holder 122 illustratively extends vertically along a side of tank 102 and illustratively has a top end 142 that in the embodiment shown in FIGS. 1-3, is above a top 144 of tank 102, but that need not be above top 144 of tank 102. In the example embodiment shown in the FIGS. 1-4, tank 102 is mounted to a table 146 with a table top 148 surrounding top 144 of tank 102 with top end 142 of UV tube holder 122 above table top 148. It should be understood that table top 148 could include a catch basin surrounding the top 144 of tank 102. Outlet 124 of UV tube holder is disposed near top end 142 of UV tube holder 122. Food waste collector system 100 also includes a controller 150 that is configured to control the controllable components of food waste collector system 100, such as pump 116, controllable valve 132, and UV tubes 136.

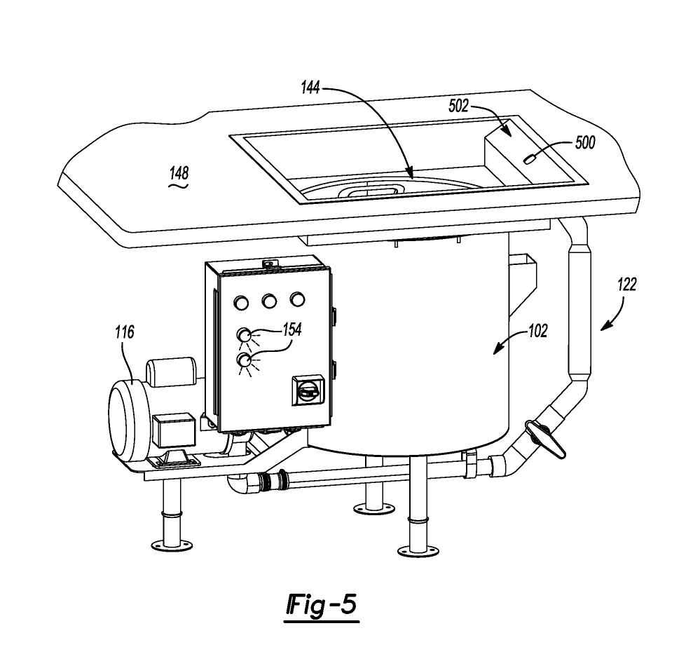

In a variation shown in FIG. 5, UV tube holder 122 does not extend above table top 148 and outlet 124 (not shown in FIG. 5) of UV tube holder 122 is coupled to a discharge outlet 500 in a tray 502 surrounding top 144 of tank 102.

In operation, water is recirculated from tank 102 by pump 116 pumping it from tank 102 through UV tube holder 122 to spout 126 where it discharged in a waterfall type discharge into perforated container 104 and tank 102. In the variation shown in FIG. 5, the water is discharged from UV tube holder 122 through discharge outlet 500 in tray 502 into tank 102. When the water flows through UV tube holder 122, bacteria in the water is sterilized by the UV light and also killed by the oxidizers emitted in the oxidation process. In the example shown in the drawings, the water flows past the UV tubes 136 disposed in UV tube holder 122. The UV light emitted by UV tubes 136 in conjunction with the catalytic surface coating 140 on inner wall 138 of UV tube holder 122 provides the oxidation process. To further reduce the possibility of contamination, in addition to the foregoing oxidation process, food waste collector system 100 periodically replaces a portion of the water being recirculated with fresh water via fresh water inlet 128.

Flow valve 133 senses whether water is flowing through it from pump 116 into UV tube holder 122. Flow valve is coupled to controller 150 as is pump 116. If flow valve 133 senses that no water is flowing through it, controller 150 then shuts pump 116 off.

In a variation, UV tube holder 122 does not having the catalytic surface coating 140 on inner wall 138. In this variation, the bacteria in the water are sterilized by the UV light but since there is no oxidation process, no oxidizers are emitted that kill the bacteria in the water.

In an example, UV tube holder 122 has a diameter of about 4 inches and a height of about thirty-eight inches and includes two UV tubes 136. It should be understood that the foregoing is an example, and UV tube holder 122 can have other dimensions and have more or fewer than two UV tubes 136.

In an illustrative sequence of operation, on start-up, controllable valve 132 is opened by controller 150 for a period of time, such as 2.5 minutes, to fill tank 102 with a desired amount of water. Thereafter, controller 150 repeatedly cycles controllable valve 132 closed and open to supply fresh water. The injection of fresh water into tank 102 causes a portion of the water in tank 102 to exit tank 102 through an overflow tube (not shown). In an example, controller 150 cycles controllable valve closed for three minutes and open for fifteen seconds at a flow rate of 2.5 gallons per minute.

Controller 150 may be or include a digital processor (DSP), microprocessor, microcontroller, or other programmable device which are programmed with software implementing the above described control of food waste collector system 100. It should be understood that other logic devices can be used, such as a Field Programmable Gate Array (FPGA), a complex programmable logic device (CPLD), or application specific integrated circuit (ASIC). When it is stated that controller 150 performs a function or is configured to perform a function, such as controlling a component of food waste collector system 100, it should be understood that controller 150 is configured to do so with appropriate logic (software, hardware, or a combination of both), such as by appropriate software, electronic circuit(s) including discrete and integrated logic, or combination thereof. Controller 150 may include a control panel enclosure 152 in which components of controller 150, such as logic devices, are disposed.

In an aspect, spout 126 may be a movable spout so that it can be moved out of the way when perforated container 104 is placed into or removed from tank 102. In an aspect, controllable valve 132 may include a mixing valve to mix hot and cold water to provide fresh water at a desired temperature, such as 107° F. Fresh water inlet 128 may then include an inlet for cold water and an inlet for hot water and controllable valve 132 may include a solenoid valve for the hot water inlet and a solenoid valve for the cold water inlet to turn the hot and cold water on and off. UV tube holder 122 includes indicator lights (not shown) that are illuminated when UV tubes 136 are illuminated. In an aspect, control panel enclosure 152 includes indicator lights 154 that are illuminated when UV tubes 136 are illuminated. In an aspect, the indicator lights, whether the indicator lights 154 and/or the indicator lights of UV tube holder 122, are connected such that if a UV tube 136 is burned out, the indicator light will be off when power is provided to the UV tube 136 to show a user that the UV tube is burned out.

In a variation, food waste collector system 100 alternatively or additionally has an ozone generator 156 (shown in phantom in FIG. 1) that injects ozone into the water recirculating in food waste collector system 100 to treat this water and kill bacteria therein. In an aspect, ozone generator 156 is coupled to tank 102 and injects ozone into the water in tank 102. It should be understood that ozone generator 156 could alternatively be coupled to other portions of food waste collector system 100, such as to the inlet or outlet of pump 116 or to UV tube holder 122. Ozone generated by ozone generator 156 is injected into the water in tank 102 to kill bacteria in the water in tank 102. It should be understood that food waste collector system 100 could have only ozone generator 156 so that the recirculating water is just treated with ozone or could have ozone generator 156 in addition to UV tube holder 122 (with or without the catalytic surface coating 140 on inner wall 138).

In an aspect, food waste collector system 100 having any one or combination of the above discussed bacteria treatment systems may also include a soap injector 155 (shown in phantom in FIG. 1) that injects soap into the recirculating water. In an aspect, soap injector 155 is coupled to tank 102 and injects soap into the water in tank 102. It should be understood that soap injector 155 could alternatively be coupled to other portions of food waste collector system 100, such as to the inlet or outlet of pump 116 or to UV tube holder 122.

In the example embodiment shown in the drawings, tank 102 may have a capacity of about 11.3 gallons and the total amount of water in use at any one time is about fourteen gallons. It should be understood that this is an example and tank 102 could have a capacity of other than about 11.3 gallons, for example 9.5 gallons, and the total amount of water in use at any one time could be other than about fourteen gallons. In an illustrative example where food waste collector system 100 has ozone injector 156 but not UV tube holder 122, the tank 102 has a capacity of about 9.5 gallons and the total amount of water in use at any one time is about 10 gallons.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A food waste collector system, comprising:
a tank;
a pump that recirculates water by pumping it from the tank and back into the tank; and
a bacteria treatment system that treats the recirculating water to reduce bacteria levels in the recirculating water.

2. The food waste collector system of claim 1, wherein the bacteria treatment system includes an ultraviolet tube holder in which at least one ultraviolet tube is disposed, the pump having an inlet coupled to an outlet of the tank and an outlet coupled to an inlet of the ultraviolet tube holder, the pump recirculating water from the tank by pumping it through the ultraviolet tube holder where it is irradiated with ultraviolet light from the ultraviolet tube to sterilize bacteria in the water and the water is then discharged from the ultraviolet tube holder to flow into a top of the tank.

3. The food waste collector system of claim 2 wherein the ultraviolet tube holder includes an inner wall with a catalytic surface that in conjunction with the ultraviolet light from the ultraviolet tube provides an oxidation process that generates oxidizers that kill the bacteria in the water.

4. The food waste collector system of claim 3 including a controllable valve coupled to a source of fresh water and controlled by a controller to cycle open and closed to inject fresh water into the water being recirculated.

5. The food waste collector system of claim 2 including a spout coupled to an outlet of the ultraviolet tube holder and through which water is discharged into the tank wherein the spout is a waterfall type spout that provides a flat, wide discharge stream of water.

6. The food waste collector system of claim 2 wherein the bacteria treatment system further includes an ozone generator that generates ozone that is injected in to the recirculating water to kill bacteria in the recirculating water.

7. The food waste collector system of claim 6 wherein the ozone generator injects the ozone into water in the tank.

8. The food waste collector system of claim 6 including a controllable valve coupled to a source of fresh water and controlled by a controller to cycle open and closed to inject fresh water into the water being recirculated.

9. The food waste collector system of claim 1 wherein the bacteria treatment system includes an ozone generator that generates ozone that is injected in to the recirculating water to kill bacteria in the recirculating water.

10. The food waste collector system of claim 9 wherein the ozone generator injects the ozone into water in the tank.

11. The food waste collector system of claim 9 including a controllable valve coupled to a source of fresh water and controlled by a controller to cycle open and closed to inject fresh water into the water being recirculated.

12. The food waste collector system of claim 1 including a controllable valve coupled to a source of fresh water and controlled by a controller to cycle open and closed to inject fresh water into the water being recirculated.

13. The food waste collector system of claim 1 and further including a soap injector that injects soap into the recirculating water.

14. The food waste collector system of claim 1 and further including a perforated container removably receivable in the tank.

15. In a food waste collector system having a tank, a pump that recirculates water by pumping it from the tank and back into the tank, a method for treating the water being recirculated comprising: treating the recirculating water to reduce bacteria in the recirculating water.

16. The method of claim 15 wherein treating the recirculating water includes pumping the water being recirculated through an ultraviolet tube holder that holds at least one ultraviolet tube and irradiating the water passing through the ultraviolet tube holder with ultraviolet light from the ultraviolet tube to sterilize bacteria in the water.

17. The method of claim 16 including periodically injecting fresh water into the recirculating water with a controllable valve coupled to a source of fresh water by cycling the controllable valve open and closed with a controller coupled to the controllable valve.

18. The method of claim 16 and further including treating the recirculating water by killing bacteria in the recirculating water by the ultraviolet light in conjunction with a catalytic surface coating of the ultraviolet tube holder providing an oxidation process that generates oxidizers that kill the bacteria in the recirculating water.

19. The method of claim 18 and further including treating the recirculating water by injecting ozone into the recirculating water to kill bacteria in the recirculating water.

20. The method of claim 19 including periodically injecting fresh water into the recirculating water with a controllable valve coupled to a source of fresh water by cycling the controllable valve open and closed with a controller coupled to the controllable valve.

21. The method of claim 15 wherein treating the recirculating water includes injecting ozone into the recirculating water to kill bacteria in the recirculating water.

22. The method of claim 21 including periodically injecting fresh water into the recirculating water with a controllable valve coupled to a source of fresh water by cycling the controllable valve open and closed with a controller coupled to the controllable valve.
